(19) United States
(12) Patent Application Publication
(10) Pub. No.: US 2006/0110729 A1
(43) Pub. Date: May 25, 2006

(54) Method of dna testing for mycobacterium paratuberculosis strains

(76) Inventor: Desmond Michael Collins, Upper Hutt (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/509,708

(22) PCT Filed: Jun. 10, 2003
(86) PCT No.: PCT/NZ03/00119

§ 371 Date: Sep. 9, 2005

(30) Foreign Application Priority Data
Jun. 10, 2002 (NZ) 519469

(51) Int. Cl. C12Q 1/68; C07H 21/04; C12N 15/74; C12N 1/21; C07K 14/35

(52) U.S. Cl. 435/6; 435/69.3; 435/252.3; 435/471; 530/350; 536/23.7

(57) ABSTRACT

The present invention relates to the discovery of a DNA sequence in sheep types of M. paratuberculosis that differs from the homologous sequence in cattle types of M. paratuberculosis. The invention also provides a nucleic acid amplification technique based on these differences that can be used to distinguish strains of the cattle type from strains of both the sheep types of M. paratuberculosis. The invention also relates to use of these sequences in a nucleic acid amplification technique to distinguish all strains of M. paratuberculosis from other strains of the MAI complex and from strains of the M. tuberculosis complex.

### TECHNICAL FIELD

This invention relates to improvements in and relating to a method of DNA testing. In particular this invention relates to nucleic acid sequences of Mycobacterium paratuberculosis and their use in a method for identifying different strains of M. paratuberculosis and distinguishing strains of M. paratuberculosis from other mycobacterial species. This invention also provides an aid in the diagnosis of diseases caused by Mycobacterial species in human and animal medical practice.

### BACKGROUND ART

Mycobacteria are rod-shaped, acid-fast, aerobic bacilli that do not form spores. A moderate number of slow-growing mycobacterial species are major pathogens for humans and/or animals. For example, paratuberculosis is a very widespread animal health problem which causes major economic losses in farming of ruminant animals particularly in the dairy industry. The development of robust diagnostic tests to distinguish different mycobacterial species and to characterise subspecies, groups and types of related strains within a species is of prime importance.

Paratuberculosis or Johne's disease is a chronic granulomatous enteritis that can affect all domestic and wild ruminants causing reduced food intake, weight loss and death. The disease is present in most countries and results in significant production losses. The causative organism, Mycobacterium avium subsp. paratuberculosis (basonym M. paratuberculosis) (Harris et al., 2001) has also been implicated as the etiologic agent of Crohn's disease in humans and is a member of the MAI complex, a group of closely related species which includes Mycobacterium intracellulare and all subspecies of M. avium. For taxonomic purposes, M. avium is divided into the three subspecies M. avium subsp. avium (although in most publications this subspecies is still referred to as M. avium), M. avium subsp. paratuberculosis and M. avium subsp. silvaticum (Thorel et al., 1990). While M. paratuberculosis appears to be an obligate pathogen, closely-related organisms of the MAI complex that share many common antigens with M. paratuberculosis are widespread throughout the environment. Exposure of animals to these environmental organisms is probably responsible for the lack of sensitivity and specificity of antigen-based diagnostic tests for M. paratuberculosis. Other problems that have made this disease particularly difficult to control are the very slow growth of the organism on artificial culture, and the ability of the organism to survive in many animals for years without causing any overt disease (Chiodini et al., 1984; Harris and Barletta, 2001).

Two recent discoveries have shown that the spread of M. paratuberculosis maybe more complicated than previously believed and emphasise the need for the development of new diagnostic tools. First, the organism has been reported to survive normal milk pasteurisation (Grant et al., 2002). Since pasteurised milk is widely consumed in many countries, this survival provides a route by which large sections of the population can be exposed to this obligate pathogen and supports the case of those who claim that it causes Crohn's disease in humans (Herron-Taylor et al., 2000; Harris and Barletta, 2001). Second, M. paratuberculosis has also been isolated in the United Kingdom from common wild non-ruminant animals such as rabbits, foxes, stoats and crows (Beard et al., 2001). This finding complicates epidemiological studies, as previously it had been believed that spread of the disease occurred only from ruminants, either directly from one animal to another or through infected milk or by grazing on pasture infected by organisms shed from another infected ruminant (Chiodini et al., 1984).

The first significant molecular biological development in the study of M. paratuberculosis was the discovery of multiple copies of an insertion sequence IS900 (Collins et al., 1989; Green et al., 1989). This sequence has been found to be specific for M. paratuberculosis and is now widely used as the basis for diagnostic tests that use DNA amplification (Collins et al., 1993; Fang et al., 2002). Related insertion sequences have been found in other members of the MAI complex (Kunze et al., 1991; Englund et al., 2002) and the finding that the most recently discovered sequence is 94% identical to IS900 has raised doubts about the specificity of tests based on parts of the IS900 sequence (Englund et al., 2002). There would be advantages in having a range of sequences that have a high probability of being specific to M. paratuberculosis so that new tests could be widely trialled to determine which sequences are truly unique to this species. Sequencing of the genomes of both an M. paratuberculosis and an M. avium subsp. avium strain is currently in progress and a range of sequences that might differ between these two strains have been identified (Bannantine, 2002). Whether all these differences are real cannot be determined until the sequencing of both genomes is completed but even then the genetic diversity of different MAI strains is such (Falkinham, 1999) that it will be some years before the degree of specificity of these sequences can be determined for a wide range of strains in the different subspecies.

Isolates of M. paratuberculosis were first characterised into cattle and sheep types in 1990 (Collins et al., 1990) on the basis of restriction fragment length polymorphisms (RFLPs) of the insertion sequence IS900 and this largely correlates with the difficulty of primary isolation of sheep types (Collins et al., 1990, Pavlik et al., 1999). The distinction into cattle and sheep types is epidemiologically useful, as cattle and sheep are preferentially infected with their named types while other ruminant species such as deer and goats appear to be infected more easily with either type (Collins et al., 1990; de Lisle et al., 1993; Pavlik et al., 1999; Whittington et al., 2000). Sheep strains from Canada (Collins et al., 1990) and subsequently from South Africa (de Lisle et al., 1992) and Iceland (de Lisle et al., 1993) were found to have RFLP patterns that clustered in a group that was different from that of cattle types and other sheep types and were classified as belonging to a third or intermediate type. A careful comparison of members of these three RFLP types revealed that the pattern of the intermediate type was more closely related to patterns of the other sheep type than to patterns of the cattle type (Pavlik et al., 1999) and for this reason, and also because of its epidemiological association with sheep, this intermediate type is better referred to as a variant or second sheep type.

At present, DNA amplification testing for paratuberculosis where both cattle and sheep types are potentially present, involves a PCR assay based on IS900 to confirm the presence of M. paratuberculosis followed by a PCR based on IS1311 whose product is then subjected to restriction endonuclease analysis (Whittington et al., 2000). This two-step PCR analysis approach is performed because IS1311 is not unique for M. paratuberculosis and is also found in M. avium subsp. avium (Collins et al., 1997), but some copies of IS1311 in M. paratuberculosis have polymorphisms that are specific for the cattle and sheep types and the polymorphisms can be detected by digesting the IS1311 PCR product with appropriate restriction enzymes (Marsh et al., 1999).

Thus, it would be useful if there could be provided a single PCR diagnostic test which can distinguish between M. paratuberculosis and other mycobacterial species of the MAI complex and also within the same test distinguish between sheep and cattle type strains of M. paratuberculosis.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### SUMMARY OF INVENTION

The present invention relates to the discovery of a DNA sequence in sheep types of M. paratuberculosis that differs from the homologous sequence in cattle types of M. paratuberculosis. The invention also provides a nucleic acid amplification technique based on these differences that can be used to distinguish strains of the cattle type from strains of both the sheep types of M. paratuberculosis. The invention also relates to use of these sequences in a nucleic acid amplification technique to distinguish all strains of M. paratuberculosis from other strains of the MAI complex and from strains of the M. tuberculosis complex.

### DISCLOSURE OF INVENTION

According to a first aspect of the present invention there is provided a nucleic acid molecule of a sheep type of M. paratuberculosis said molecule comprising SEQ ID NO. 1 or a complement thereof.

According to a second aspect of the present invention there is provided a probe comprising SEQ ID NO. 1 or a complement thereof.

According to a third aspect of the present invention there is provided a probe comprising at least 6 contiguous nucleotides selected from nucleotides 1-35 of SEQ ID NO. 1 or a complement thereof.

Preferably, the probe substantially as described above may include at least 10-12 contiguous nucleotides selected from nucleotides 1-35 of SEQ ID NO. 1 or a complement thereof.

More preferably the probe substantially as described above may include more than 20 contiguous nucleotides selected from nucleotides 1-35 of SEQ ID NO. 1 or a complement thereof.

According to a fourth aspect of the present invention there is provided a probe comprising at least 6 contiguous nucleotides selected from nucleotides 230-260 of SEQ ID NO. 1 or a complement thereof.

Preferably the probe substantially as described above may include 10-12 contiguous nucleotides selected from nucleotides 230-260 of SEQ ID NO. 1 or a complement thereof.

More preferably the probe substantially as described above may include more than 20 contiguous nucleotides selected from nucleotides 230-260 of SEQ ID NO. 1 or a complement thereof.

According to a fifth aspect of the present invention there is provided a use of a nucleic acid molecule or probe substantially as described above for detecting the presence of sheep types of M. paratuberculosis.

According to a sixth aspect of the present invention there is provided a use of SEQ ID NO 2 or, a fragment or a complement, thereof for detecting the presence of cattle types of M. paratuberculosis.

According to a seventh aspect of the present invention there is provided a method of distinguishing between cattle and sheep types of M. paratuberculosis comprising the step of comparing differences between the nucleotide sequences of SEQ ID NO. 1 and SEQ ID NO. 2 or complements of said sequences.

According to a eighth aspect of the present invention there is provided a method of detecting the presence of M. paratuberculosis in a sample via a nucleic acid amplification technique said method comprising the steps of:
a) taking a sample from an animal or any other source;
b) extracting nucleic acids from the sample or culturing mycobacteria from the sample and extracting nucleic acids from the mycobacterial culture;
c) performing a nucleic acid amplification technique; and
d) determining the identity of the amplification product.

Preferably animals may include cattle, sheep, deer, goats, ferrets, rabbits and humans.

According to a ninth aspect of the present invention there is provided a method substantially as described above wherein step d) of the method comprises identifying the presence of at least 6 nucleotides of the nucleic acid molecule comprising SEQ ID NO. 1 or a complement thereof.

According to a tenth aspect of the present invention there is provided a method substantially as described above wherein step d) of the method comprises identifying the presence of 10-12 contiguous nucleotides of the nucleic acid molecule comprising SEQ ID NO. 1 or a complement thereof.

According to an eleventh aspect of the present invention there is provided a method substantially as described above wherein step d) of the method comprises identifying the presence of at least 15 contiguous nucleotides of the nucleic acid molecule comprising SEQ ID NO. 1 or a complement thereof.

According to a twelfth aspect of the present invention there is provided a method substantially as described above wherein step d) of the method comprises identifying the presence of substantially 20 contiguous nucleotides of the nucleic acid molecule comprising SEQ ID NO. 1 or a complement thereof.

According to a thirteenth aspect of the present invention there is provided a method substantially as described above wherein step c) utilizes one oligonucleotide primer complementary to at least 6 contiguous nucleotides of SEQ ID NO. 1 or a complement thereof; and one oligonucleotide primer complementary to at least 6 nucleotides of IS900 or a complement thereof.

According to a fourteenth aspect of the present invention there is provided a method substantially as described above wherein step c) utilizes one oligonucleotide primer complementary to 10-12 contiguous nucleotides of SEQ ID NO. 1 or a complement thereof and one oligonucleotide primer complementary to 10-12 nucleotides of IS900 or a complement thereof.

According to a fifteenth aspect of the present invention there is provided a method substantially as described above wherein step c) utilizes one oligonucleotide primer complementary to substantially 15 contiguous nucleotides of SEQ ID NO. 1 or a complement thereof and one oligonucleotide primer complementary to substantially 15 nucleotides of IS900 or a complement thereof.

According to a sixteenth aspect of the present invention there is provided a method substantially as described above wherein step c) utilizes one oligonucleotide primer complementary to substantially 20 contiguous nucleotides of SEQ ID NO. 1 or a complement thereof; and one oligonucleotide primer complementary to substantially 20 nucleotides of IS900 or a complement thereof.

According to a seventeenth aspect of the present invention there is provided a method substantially as described above wherein step c) of the method comprises identifying the presence of at least 6 contiguous nucleotides of the nucleic acid molecule comprising SEQ ID NO. 2 or a complement thereof.

According to an eighteenth aspect of the present invention there is provided a method substantially as described above wherein step d) of the method comprises identifying the presence of 10-12 contiguous nucleotides of the nucleic acid molecule comprising SEQ ID NO. 2 or a complement thereof.

According to a nineteenth aspect of the present invention there is provided a method substantially as described above wherein step d) of the method comprises identifying the presence of at least 15 contiguous nucleotides of the nucleic acid molecule comprising SEQ ID NO. 2 or a complement thereof.

According to a twentieth aspect of the present invention there is provided a method substantially as described above wherein step d) of the method comprises identifying the presence of approximately 20 contiguous nucleotides of the nucleic acid molecule comprising SEQ ID NO. 2 or a complement thereof.

According to a twenty-first aspect of the present invention there is provided a method substantially as described above wherein step c) utilizes one oligonucleotide primer complementary to at least 6 contiguous nucleotides of SEQ ID NO. 2 or a complement thereof; and one oligonucleotide primer complementary to at least 6 nucleotides of IS900 or a complement thereof.

According to a twenty-second aspect of the present invention there is provided a method substantially as described above wherein step c) utilizes one oligonucleotide primer complementary to 10-12 contiguous nucleotides of SEQ ID NO. 2 or a complement thereof; and one oligonucleotide primer complementary to 10-12 nucleotides of IS900 or a complement thereof.

According to a twenty-third aspect of the present invention there is provided a method substantially as described above wherein step c) utilizes one oligonucleotide primer complementary to substantially 15 contiguous nucleotides of SEQ ID NO. 2 or a complement thereof; and one oligonucleotide primer complementary to substantially 15 nucleotides of IS900 or a complement thereof.

According to a twenty-fourth aspect of the present invention there is provided a method substantially as described above wherein step c) utilizes one oligonucleotide primer complementary to substantially 20 contiguous nucleotides of SEQ ID NO. 2 or a complement thereof; and one oligonucleotide primer complementary to substantially 20 nucleotides of IS900 or a complement thereof.

According to a twenty-fifth aspect of the present invention there is provided a use of a probe comprising at least 6 contiguous nucleotides selected from the nucleic acid comprising SEQ ID NO. 2 or a complement thereof.

According to a twenty-six aspect of the present invention there is provided a use of a probe comprising substantially 10-12 contiguous nucleotides selected from the nucleic acid comprising SEQ ID NO. 2 or a complement thereof.

According to a twenty-seventh aspect of the present invention there is provided a use of a probe comprising at least 15 contiguous nucleotides selected from the nucleic acid comprising SEQ ID NO. 2 or a complement thereof.

According to a twenty-eighth aspect of the present invention there is provided a use of a probe comprising at least 20 contiguous nucleotides selected from the nucleic acid comprising SEQ ID NO. 2 or a complement thereof.

According to a twenty-ninth aspect of the present invention there is provided use of SEQ ID NO.1 and/or SEQ ID NO. 2, or a fragment or complement thereof, substantially as described above to determine whether a strain of either a sheep type or a cattle type of M. paratuberculosis is present in a sample.

According to a thirtieth aspect of the present invention there is provided a use of SEQ ID NO.1, or a fragment or complement thereof, to distinguish any strain of M. paratuberculosis from any other strain of the MAI complex which may be present in a sample.

According to a thirty-first aspect of the present invention there is provided a use of SEQ ID NO.2, or a fragment or complement thereof, above to distinguish any strain of M. paratuberculosis from any other strain of the MAI complex which may be present in a sample.

According to a thirty-second aspect of the present invention there is provided a use of SEQ ID NO.1, or a fragment or complement thereof to distinguish any strain of M. paratuberculosis from any strain of the M. tuberculosis complex which may be present in a sample.

According to a thirty-third aspect of the present invention there is provided a use of SEQ ID NO.2, or a fragment or complement thereof, to distinguish any strain of M. paratuberculosis from any strain of the M. tuberculosis complex which may be present in a sample.

According to a thirty-fourth aspect of the present invention there is provided a use of SEQ ID NO. 1, or a fragment or complement thereof to detect the presence of M. paratuberculosis as a causative agent of Johne's disease or Crohn's disease.

According to a thirty-fifth aspect of the present invention there is provided a use of SEQ ID NO. 2, or a fragment or complement thereof, to detect the presence of M. paratuberculosis as a causative agent of Johne's disease or Crohn's disease.

It will be appreciated by those skilled in the art such that know and use of the nucleotide sequences of SEQ ID NO. 1 and SEQ ID NO. 2 will be useful as an aid in the diagnosis of these diseases.

The term “sheep type of M. paratuberculosis” as used herein refers to a strain of M. paratuberculosis which preferentially infects sheep but also may infect other species for example deer, goats and humans but does not preferentially infect cattle.

The term “cattle type of M. paratuberculosis” as used herein refers to a strain of M. paratuberculosis which preferentially infects cattle but also may infect other species for example deer, goats and humans but does not preferentially infect sheep.

The term “IS900” as used herein refers to a known DNA sequence that is characteristically present in strains of M. paratuberculosis and which is currently used to detect M. paratuberculosis species.

“Probes” are single-stranded nucleic acid molecules with known nucleotide sequences which are labelled in some way (for example, radioactively, fluorescently or immunologically), which are used to find and mark a target DNA or RNA sequence by hybridizing to it.

“Primers” are short nucleic acids, preferably DNA oligonucleotides 15 nucleotides or more in length, which are annealed to a complementary target DNA strand by nucleic acid hybridization to form a hybrid between the primer and the target DNA strand; they can then be extended along the target DNA strand by a polymerase, preferably a DNA polymerase. Primer pairs can be used for amplification of a nucleic acid sequence, e.g. by the polymerase chain reaction (PCR) or other nucleic acid amplification methods well known in the art. PCR-primer pairs can be derived from the sequence of a nucleic acid according to the present invention, for example, by using computer programs intended for that purpose such as Primer (Version 0.5© 1991, Whitehead Institute for Biomedical Research, Cambridge, Mass.).

A fragment of a nucleic acid is a portion of the nucleic acid that is less than full length and comprises at least a minimum sequence capable of hybridising specifically with a nucleic acid molecule according to the present invention (or a sequence complementary thereto) such that the fragment can have at least one of the utilities of the nucleic acid of the present invention.

The term “complement” refers to a second single stranded nucleic acid molecule having a nucleotide sequence corresponding to the nucleotide sequence of a first nucleic acid molecule: as determined by the base pairing of adenosine to Thymine and of guanine to cytosine as occurs in a double stranded DNA molecule.

The term “nucleic acid amplification technique” as used herein may generally be considered to refer to the polymerase chain reaction or PCR. However, it may equally refer to other equivalent techniques for amplifying nucleic acids known to those skilled in the art.

The term “polymerase chain reaction or PCR” as used herein refers to a system for in vitro amplification of DNA. Two synthetic oligonucleotide primers, which are complementary to two regions of the target DNA (one for each strand) to be amplified, are added to the target DNA (that need not be pure), in the presence of excess deoxynucleotides and Taq polymerase, a heat-stable DNA polymerase. In a series of temperature cycles, the target DNA is repeatedly denatured, annealed to the primers (typically at 50-60° C.) and a daughter strand extended from the primers. As the daughter strands themselves act as templates for subsequent cycles, DNA fragments matching both primers are amplified exponentially.

The nucleic acid molecule may be an RNA, cRNA, genomic DNA or cDNA molecule, and may be single- or doublestranded. The nucleic acid molecule may also optionally comprise one or more synthetic, non-natural or altered nucleotide bases, or combinations thereof. The detection of the amplified nucleic acid may be by any of a wide range of techniques known to those skilled in the art, including but not limited to size separation techniques such as gel electrophoresis, probe detection systems either on solid supports or in solution and DNA microarray techniques.

### BRIEF DESCRIPTION OF DRAWINGS

Further aspects of the present invention will become apparent from the following description which is given by way of example only and with reference to the accompanying drawings in which:

FIG. 1 Shows nucleic acid SEQ ID NO. 1;

FIG. 2 Shows nucleic acid SEQ ID NO 2;

FIG. 3 Shows PCR products from DNA of two sheep types and two cattle types of M. paratuberculosis performed with oligonucleotide primers DMC136 and DMC137 at an annealing temperature of 50° C.;

FIG. 4 Shows Alignment of homologous DNA sequences from cattle and sheep types of M. paratuberculosis. Identical nucleotides in both sequences are shaded, arrows indicate the identity and direction of the oligonucleotide primers used, the tandem DNA sequence present in the sheep type is shown in boxes, the pallindromic region is underlined, the complement of the 5′ end of the coding sequence of the putative gene involved in phage attachment is shown in lower case text, and dots indicate no sequence;

FIG. 5 Shows Diagrammatic representation, relative to the chromosome of the cattle type, of the position of SEQ ID NO. 1 and the points of insertion of the tandem repeat and the likely copy of IS900 in the sheep type; and

FIG. 6 PCR products from cattle and sheep types of M. paratuberculosis amplified with the three oligonucleotide primers DMC529, DMCS31, and DMC533. Lanes: 1 and 11, molecular size markers; 2-5, cattle types; 6-9, sheep types; 10, negative control.

FIG. 7 PCR products from cattle types of M. paratuberculosis amplified with the three oligonucleotide primers DMC529, DMC531, and DMC533 from BACTEC 12B radiometric medium containing egg yolk. Lanes: 1-3, BACTEC cultures of M. paratuberculosis; 4, positive control DNA from cattle type of M. paratuberculosis; 5, negative control; 7 molecular size markers.

### BEST MODES FOR CARRYING OUT THE INVENTION

Experimental

Non-limiting examples illustrating the invention will now be provided. It will be appreciated that the above description is provided by way of example only and variations in the materials and technique used which are known to those skilled in the art are contemplated.

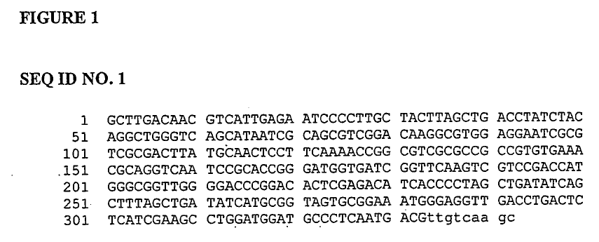

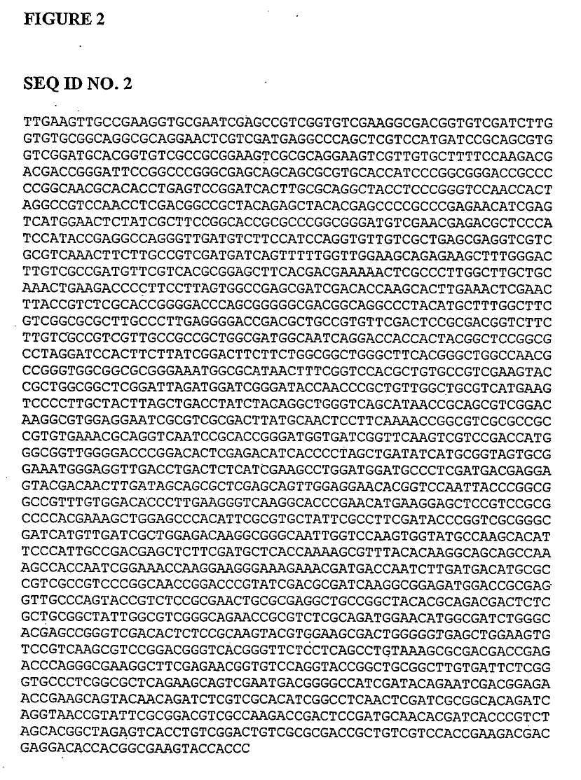

The present invention provides a DNA sequence, SEQ ID NO. 1 (FIG. 1), that is unique to sheep types of M. paratuberculosis and provides the use of SEQ ID NO. 2 (FIG. 2) for diagnostic testing for organisms of the MAI complex. The present invention also provides for the specific use of SEQ ID NO. 1 and SEQ ID NO. 2 to distinguish between sheep types and cattle types of M. paratuberculosis and to distinguish all M. paratuberculosis strains from other strains of the closely related MAI complex and from strains of the M. tuberculosis complex. A PCR diagnostic test using three oligonucleotide primers is given as an example of the utility of the invention but the invention is not limited to these oligonucleotides or to the use of PCR and a wide range of other diagnostic tests based on these sequences, their complements or any RNA or protein that they specify is envisaged.

Discovery of SEQ ID NO. 1

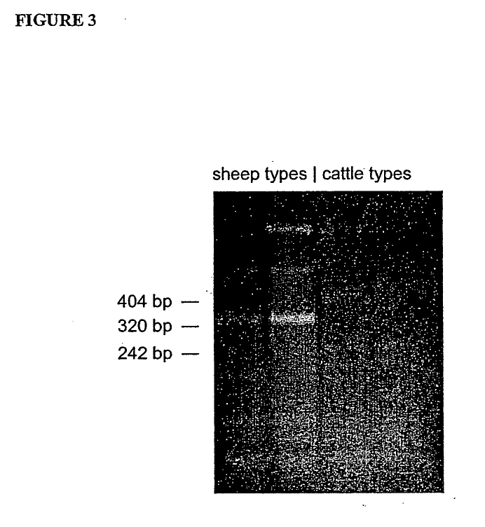

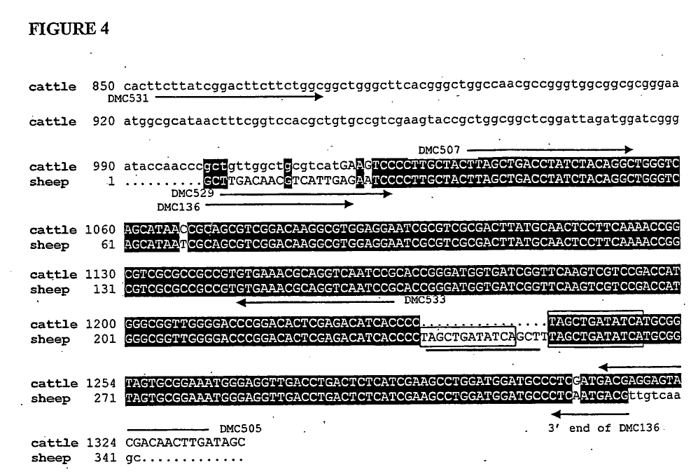

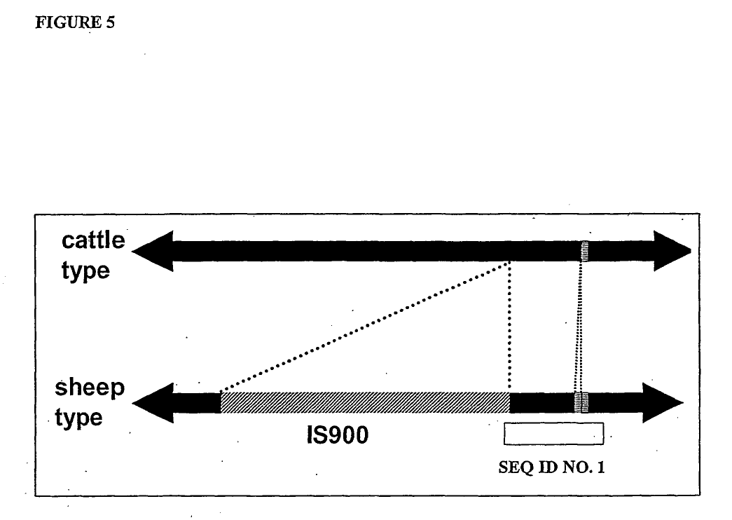

The experimental work described here is also summarised in Collins et al., (2002). The strains of the MAI complex used in this work are given in Table 1. Oligonucleotide primers used in the work are given in Table 2. Strains were cultured using specialised mycobacterial media (Collins et al., 1997; Whittington et al., 1999). Purified DNA was extracted as described previously (Collins et al., 1990). When DNA from strains of both the sheep and cattle types of M. paratuberculosis was subjected to PCR at an annealing temperature of 50° C. using primers DMC136 and DMC137 directed outwards from each end of IS900, only DNA from sheep types gave a major product between 300 bp and 400 bp (FIG. 3). Subsequently, it was observed that the same 342 bp product was obtained if only one PCR primer (DMC136) was used. The PCR product was extracted from the gel, re-amplified at an annealing temperature of 65° C., cloned into pBluescript KSII (Stratagene) and sequenced (FIG. 1). Comparison of this sequence using the programme BLAST against the partially completed sequence of the genome of a cattle type of M. paratuberculosis (National Centre for Biotechnology Information database [http://www.ncbi.nlm.nih.gov/]) indicated a high degree of homology to positions 1020-1316 of SEQ ID NO. 2 (FIG. 2), denoted as contig 249 in the database. Comparisons using the GAP programme of GCG (Wisconsin Package Version 10.2, Genetics Computer Group, Madison, Wis.) delineated two major differences (FIG. 4). First, the sheep type but not the cattle type has a tandem repeat of a 12 bp sequence followed by a 4 bp linker that together contain a 14 bp pallindromic sequence. Second, the cattle type was not homologous to DMC136 at the 5′ end. This indicated that an IS900 element was inserted at the 5′ end of SEQ ID NO. 1 in the sheep type but not in the homologous region of SEQ ID NO. 2 in the cattle type. Further investigation using DMC505 and DMC507 indicated that both types have similar sequences at the 3′ end because these primers worked equally well in a PCR reaction with an annealing temperature of 60° C. on DNA from both sheep and cattle types and gave products differing by only 16 bp. It thus appears that the product shown in FIG. 3 was produced by using the single primer DMC136 because this primer is completely homologous to DNA of the ovine type at the 5′ end of SEQ ID NO. 1 (FIGS. 1 and 4) but homologous to only about the last 10 nucleotides at the 3′ end. For this reason, the final nine nucleotides of SEQ ID NO. 1 (FIG. 1) are shown in lower case text. The most likely explanation for homology of the 5′ end of SEQ ID NO. 1 to DMC136 is that a copy of IS900 is inserted at this position in the genome of sheep types but not cattle types and this is shown in diagrammatic form in FIG. 5. Confirmation of the presence of this copy of IS900 was provided by performing a PCR on both sheep and cattle types of M. paratuberculosis using the oligonucleotide primers DMC137, which reads out of IS900 from the opposite end to DMC136, and DMC531 which would be expected to read towards the DMC137 end of IS900 in sheep types of M. paratuberculosis. As expected, this pair of primers gave a product of the expected size with sheep types of M. paratuberculosis but no product with cattle types. Clearly, to one with skill in the art this provides alternative regions of IS900 and SEQ ID NO. 2 to those used in the PCR example below and these alternative regions could be used for designing oligonucleotide primers and constructing PCR tests that potentially have similar utility to that of the example. Further analysis of SEQ ID NO. 1 and SEQ ID NO 2 using other GCG programmes showed that both the tandem repeat and the difference between sheep and cattle types at the 5′ end of SEQ ID NO. 1 are in or adjacent to likely coding sequences one of which has high homology to a gene whose product is involved in phage attachment (Barsom and Hatfull, 1996). These differences may therefore be important in determining the host preference of sheep and cattle types. If this is the case, these DNA differences observed between cattle and sheep types may be a very widespread or even ubiquitous phenomenon. Comparison of SEQ ID NO. 1 and SEQ ID NO 2 (FIGS. 1 and 2) to the incomplete genome sequence of an M. avium subsp. avium strain (National Centre for Biotechnology Information database [http://www.ncbi.nlm.nih.gov/]) did not identify any closely homologous sequences apart from the first 285 bp of SEQ ID NO 2. This indicated that SEQ ID NO. 1 and most of SEQ ID NO. 2 might not be present in M. avium subsp. avium and that these sequences could thus be used for constructing tests to distinguish between M. paratuberculosis and M. avium subsp. avium.

Development of a PCR Assay

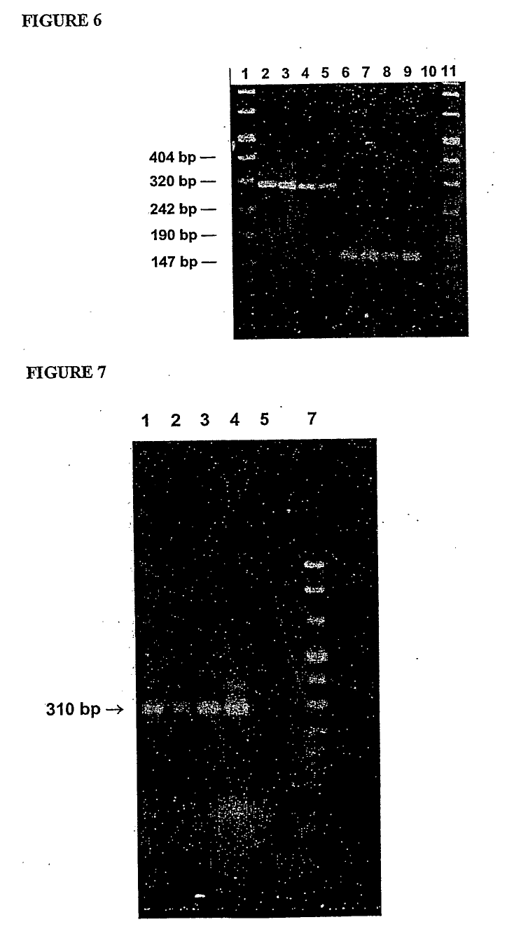

A PCR assay was developed using a GeneAmp PCR System 9600 (Applied Biosystems) and the three primers DMC529, DMC531, DMCS33 (Table 2 and FIG. 4) under the following conditions: 1 cycle at 95° C., 3 min; 25 cycles at 60° C., 30 s, 72° C., 30 s, 94° C., 30 s; 1 cycle at 72° C., 7 min. DNA from all 19 strains of the cattle type (Table 1) gave the expected product of 310 bp, while DNA from all 12 strains of the sheep type (Table 1) gave the expected product of 162 bp (FIG. 6). A PCR product was not observed for any of the wide range of strains of the MAI complex (Table 1) that did not contain IS900 and were not M. paratuberculosis. No PCR product was observed with strains of Mycobacterium tuberculosis and Mycobacterium bovis (Table 1) which are members of the M. tuberculosis complex. This group of organisms which cause tuberculosis in mammals comprise the following species: M. tuberculosis, M. bovis, Mycobacterium bovis subsp. caprae, Mycobacterium microti and Mycobacterium canettii. M. tuberculosis causes most of the tuberculosis in humans and M. bovis causes tuberculosis in a wide range of mammals including humans, cattle and deer. In some situations there is diagnostic utility in having a fast diagnostic test such as PCR to distinguish between samples from animals infected with strains of the M. tuberculosis complex and those infected with M. paratuberculosis (de Lisle et al., 1993).

The MAI complex covers a relatively broad group of genetically related mycobacteria that, with the exception of M. paratuberculosis, are found in many environmental niches and are occasional mammalian pathogens. Because of the potential of these organisms to confuse the diagnosis of paratuberculosis, strains of the MAI complex tested in this study were weighted towards those that had been isolated from humans or from a range of different animal hosts and that might be expected to be most closely related to M. paratuberculosis (Collins et al., 1997). The fact that none of these strains was positive in the PCR assay gives a high level of confidence in the utility of the test to distinguish between strains of M. paratuberculosis and other strains of the MAI complex. In the case of M. paratuberculosis, the inclusion of 10 strains from five other countries, including sheep strains from Canada, South Africa and Iceland, enabled a cross-section of strains with different IS900 RFLP types to be tested. In all cases, the PCR results were consistent with this RFLP division into sheep and cattle types, indicating that tests based on SEQ ID NO. 1 and SEQ ID NO. 2 should have wide utility for distinguishing between sheep and cattle types of M. paratuberculosis in many countries. Because of the association of M. paratuberculosis with Crohn's disease in humans, assays based on SEQ ID NO. 1 and SEQ ID NO. 2 not only have the potential to be widely applicable to epidemiological and other studies of paratuberculosis but will also have utility in the field of Crohn's disease.

Detection of M. paratuberculosis in Modified BACTEC Medium

For detection of M. paratuberculosis in modified BACTEC 12B radiometric medium containing egg yolk (Whittington et al., 1999), 200 μl of medium containing the organisms was added to approximately 0.5 ml glass beads (Qbiogene Lysing Matrix B) and extracted using QIAamp DNA Stool Mini Kit (QIAGEN) by treating the sample plus added glass beads as if it was a stool sample and following the manufacturer's protocols with minor modifications as outlined. The sample plus added glass beads was vortexed in 1 ml proprietary ASL buffer from the QIAamp kit for two periods of 20 sec each in a Ribolyser (ThermoSavant FastPrep Cell Disrupter) set on 6.5 with 1 min cooling on ice between each period. The suspension was heated at 95° C. for 10 min, vortexed for 15 sec and centrifuged. Approximately 800 μl of supernatant was removed, made up to 1.2 ml with proprietary ASL buffer and treated from then on as detailed in the Manufacturer's protocols. Briefly, the 1.2 ml sample was vortexed with an InhibitEX tablet and incubated for 1 min at room temperature. After centrifuging twice, 200 μl of supernatant was added to 15 μl Proteinase K, 200 μl proprietary AL buffer was added and the mixture was vortexed for 15 sec and then incubated at 70° C. for 10 min. The mixture was vortexed with 200 μl absolute ethanol and eluted through a QIAamp (QUIGEN) spin column as detailed in the Manufacturer's protocols. A final eluate of approximately 200 μl was collected and 10-20 μl was subjected to PCR with the three primers DMC529, DMC531, DMC533. The PCR conditions used were either 1 cycle at 95° C., 3 min; 42 cycles at 50° C., 30 s, 72° C., 30 s, 94° C., 30 s; 1 cycle at 72° C., 7 min; or 1 cycle at 95° C., 3 min; 42 cycles at 45° C., 30 s, 72° C., 30 s, 94° C., 30 s; 1 cycle at 72° C., 7 min. PCR products using the last set of PCR conditions above for cattle types of M. paratuberculosis cultured in BACTEC medium containing egg yolk are shown in FIG. 7.

TABLE 1
Strains of the MAI complex and M. tuberculosis complex subjected to
PCR

| No. of | With | With | | |
|---|---|---|---|---|
| strains | IS900 | IS901 | Description | Source |
| 10 | 0 | 3 | Reference serotypes 1-6, 8-11 | Dawson** |
| | | | of MAI complex* | |
| 11 | 0 | 7 | Cattle, deer and pig isolates | New |
| | | | of MAI complex* | Zealand |
| 6 | 0 | 5 | Bird isolates of MAI complex* | New |
| | | | | Zealand |
| 4 | 0 | 0 | Human isolates of MAI complex* | New |
| | | | | Zealand |
| 14 | 14 | — | M. paratuberculosis cattle type | New |
| | | | | Zealand |
| 3 | 3 | — | M. paratuberculosis cattle type | Canada |
| 1 | 1 | — | M. paratuberculosis TMC1613; | USA |
| | | | cattle type | |
| 1 | 1 | — | M. paratuberculosis 316F; cattle | UK |
| | | | type | |
| 7 | 7 | — | M. paratuberculosis sheep type | New |
| | | | | Zealand |
| 1 | 1 | — | M. paratuberculosis sheep type | Canada |
| 3 | 3 | — | M. paratuberculosis sheep type | South Africa |
| 1 | 1 | — | M. paratuberculosis sheep type | Iceland |
| 1 | 1 | — | M. tuberculosis H37Rv | USA |
| 2 | — | — | M. bovis | New |
| | | | | Zealand |

*None of these strains of the MAI complex were M. paratuberculosis
**See Wards et al. (1987)

TABLE 2

DNA oligonucleotide primers
used in this work

| | |
|---|---|
| Oligonucleotide | Sequence 5′ - 3′ |

| | |
|---|---|
| DMC136 | GCTTGACAACGTCATTGAG |
| DMC137 | CCCTTCAAGAAAGGTAAGG |
| DMC505 | CAAGTTGTCGTACTCCTCGTC |
| DMC507 | TTAGCTGACCTATCTACAGGC |
| DMC529 | TTGACAACGTCATTGAGAATCC |
| DMC531 | TCTTATCGGACTTCTTCTGGC |
| DMC533 | CGGATTGACCTGCGTTTCAC |

Aspects of the present invention have been described by way of example only and it should be appreciated that modifications and additions may be made thereto without departing from the scope thereof as defined in the appended claims.

### REFERENCES

Bannantine, J. P., Baechler, E., Zhang, Q., Li, L., Kapur, V. (2002) Genome Scale Comparison of Mycobacterium avium subsp. paratuberculosis with Mycobacterium avium subsp. avium reveals potential diagnostic sequences. J Clin Microbiol. 40: 1303-1310.
Barsom, E. K., and G. F. Hatfull. (1996) Characterization of Mycobacterium smegmatis gene that confers resistance to phages L5 and D29 when overexpressed. Mol. Microbiol. 21:159-170.
Beard, P. M., M. J. Daniels, D. Henderson, A. Pirie, K. Rudge, D. Buxton, S. Rhind, A. Greig, M. R. Hutchings, I. McKendrick, K. Stevenson, and J. M. Sharp. (2001) Paratuberculosis infection of nonruminant wildlife in Scotland. J Clin Microbiol 39:1517-1521.
Chiodini, R. J., Van Kruiningen, H. J., Merkal, R. S. (1984) Ruminant paratuberculosis (Johne's disease): the current status and future prospects. Cornell Vet 74: 218-262.
Collins, D. M., Gabric, D. M., de Lisle, G. W. (1989) Identification of a repetitive DNA sequence specific to Mycobacterium paratuberculosis. FEMS Microbiol Lett 60: 175-178.
Collins, D. M., Gabric, D. M., de Lisle, G. W. (1990) Identification of two groups of Mycobacterium paratuberculosis strains by restriction endonuclease analysis and DNA hybridization. J Clin Microbiol 28: 1591-1596.
Collins, D. M., Stephens, D. M., de Lisle, G. W. (1993) Comparison of polymerase chain reaction tests and faecal culture for detecting Mycobacterium paratuberculosis in bovine faeces. Vet Microbiol 36: 289-299.
Collins, D. M., Cavaignac, S., de Lisle, G. W. (1997) Use of four DNA insertion sequences to characterise strains of the Mycobacterium avium complex isolated from animals. Mol Cell Probes 11: 373-380.
Collins, D. M., De Zoete, M., Cavaignac, S. M. (2002) Mycobacterium avium subsp. paratuberculosis from cattle and sheep can be distinguished by PCR based on a novel DNA sequence difference. J Clin Microbiol 40: 4760-4762.
de Lisle, G. W., Collins, D. M., Huchzermeyer, H. F. A. K. (1992) Characterization of ovine strains of Mycobacterium paratuberculosis by restriction endonuclease analysis and DNA hybridization. Onderstepoort J Vet Res 59: 163-165.
de Lisle, G. W., Yates, G. F., Collins, D. M. (1993) Paratuberculosis in farmed deer; case reports and DNA characterization of isolates of Mycobacterium paratuberculosis. J Vet Diagn Invest 5: 567-571.
Englund, S., Bolske, G., Johansson, K. E. (2002) An IS900-like sequence found in a Mycobacterium sp. other than Mycobacterium avium subsp. paratuberculosis. FEMS Microbiol Lett 209: 267-271.
Falkinham, J. O. (1999) Molecular epidemiology: other mycobacteria in Mycobacteria: molecular biology and virulence, Ratledge, C. R., Dale, J. (eds), Blackwell Science, London, pp. 136-160.
Fang, Y., Wu, W. H., Pepper, J. L., Larsen, J. L., Marras, S. A., Nelson, E. A., Epperson, W. B., Christopher-Hennings, J. (2002) Comparison of real-time, quantitative PCR with molecular beacons to nested PCR and culture methods for detection of Mycobacterium avium subsp. paratuberculosis in bovine fecal samples. J Clin Microbiol. 40: 287-291.
Grant, I. R., Hitchings, E. I., McCartney, A., Ferguson F., Rowe M. T. (2002) Effect of commercial-scale high-temperature, short-time pasteurization on the viability of Mycobacterium paratuberculosis in naturally infected cows' milk. Appl Envir Microbiol 68: 602-607.
Green, E. P., Tizard, M. L., Moss, M. T., Thompson, J., Winterbourne, D. J., McFadden, J. J., Hennon-Taylor, J. (1989) Sequence and characteristics of IS900, an insertion element identified in a human Crohn's disease isolate of Mycobacterium paratuberculosis. Nucleic Acids Res 17: 9063-9073.
Harris, N. B., Barletta, R. G. (2001) Mycobacterium avium subsp. paratuberculosis in veterinary medicine. Clin Adicrobiol Rev 14: 489-512.
Hermon-Taylor, J., Bull, T. J., Sheridan, J. M., Cheng, J., Stellakis, M. L., Sumar, N. (2000) Causation of Crohn's disease by Mycobacterium avium subspecies paratuberculosis. Can J Gastroenterol 14: 521-539.
Kunze, Z. M., Wal, S., Appelberg, R., Silva, M. T., Portaels, F., McFadden, J. J. (1991) IS901, a new member of a widespread class of atypical insertion sequences, is associated with pathogenicity in Mycobacterium avium. Mol Microbiol 5: 2265-2272.
Marsh, I., Whittington, R., Cousins, D. (1999) PCR-restriction endonuclease analysis for identification and strain typing of Mycobacterium avium subsp. paratuberculosis and Mycobacterium avium subsp. avium based on polymorphisms in IS1311. Mol Cell Probes 13: 115-126.
Pavlik, I., Horvathova, A., Dvorska, L., Bartl, J., Svastova, P. du Maine, R., Rychlik, I. (1999) Standardisation of restriction fragment length polymorphism analysis for Mycobacteriun avium subspecies paratuberculosis. J Microbiol Methods 38: 155-167.
Thorel, M. F., Krichevsky, M., Levy-Frebault, V. V. (1990) Numerical taxonomy of mycobactin-dependent mycobacteria, emended description of Mycobacterium avium, and description of Mycobacterium avium subsp. avium subsp. nov., Mycobacterium avium subsp. paratuberculosis subsp. nov., and Mycobacterium avium subsp. silvaticum subsp. nov. Int J Syst Bacteriol 40: 254-260.
Wards, B. J., Collins, D. M., de Lisle, G. W. (1987) Restriction endonuclease analysis of members of the Mycobacteriuni avium-M. intracellulare-M. scrofulaceum serocomplex. J Clin Microbiol 25: 2309-2313.
Whittington, R J, Marsh, I., McAllister, S., Turner, M. J., Marshal, D. J., Fraser, C. A (1999) Evaluation of modified BACTEC 12B radiometric medium and solid media for culture of Mycobacterium avium subsp. paratuberculosis from sheep. J Clin Microbiol 37: 1077-1083
Whittington, R. J., Hope, A. F., Marshall, D. J., Taragel, C. A, Marsh, I. (2000) Molecular epidemiology of Mycobacterium avium subsp. paratuberculosis: IS900 restriction fragment length polymorphism and IS1311 polymorphism analyses of isolates from animals and a human in Australia. J Clin Microbiol 38: 3240-3248.

1. An isolated nucleic acid molecule of a sheep type of M. paratuberculosis said molecule comprising SEQ ID NO:1 or a complement thereof.

2. The nucleic acid molecule of claim 1 wherein said molecule is a probe.

3. An isolated nucleic acid molecule comprising at least 20 or more contiguous nucleotides selected from the group consisting of nucleotides 230-260 of SEQ ID NO: 1 and a complement thereof.

4. A method for detecting the presence of sheep types of M. paratuberculosis comprising, identifying the presence of at least 20 or more contiguous nucleotides of SEQ ID NO:1 or a complement thereof in the sample.

5. A method for detecting the presence of cattle types of M. paratuberculosis comprising, identifying the presence of at least 20 or more contiguous nucleotides of SEQ ID NO:2 or a complement thereof in the sample.

6. A method of distinguishing between cattle and sheep types of M. paratuberculosis comprising the step of identifying the presence of the nucleotide sequences of SEQ ID NO.1 and SEQ ID NO. 2 or complements of said sequences, wherein homology to SEQ ID NO:1 indicates that the M. paratuberculosis is of the cattle type and homology to SEQ ID NO:2 indicates that the M. paratuberculosis is of the sheep type.

7. A method of detecting the presence of M. paratuberculosis in a sample via a nucleic acid amplification technique said method comprising the steps of:
a) taking a sample from an animal or any other source;
b) extracting nucleic acids from the sample or culturing mycobacteria from the sample and extracting nucleic acids from the mycobacterial culture;
c) performing a nucleic acid amplification technique with one or more nucleic acid sequences wherein said amplification techniques identifies the presence of at least 10-12 contiguous nucleotides of the nucleic acid molecule comprising SEQ ID NO. 1 or a complement thereof; and
d) determining the presence of M. paratuberculosis by the presence of an amplification product.

8. A method as claimed in claim 7, wherein the animals are selected from the group consisting of: cattle, sheep, deer, goats, ferrets, rabbits and humans.

9. (canceled)

10. A method claimed in claim 7, wherein step d) of the method comprises identifying the presence of at least 15 contiguous nucleotides of the nucleic acid molecule comprising SEQ ID NO. 1 or a complement thereof.

11. A method as claimed in claim 7, wherein step d) of the method comprises identifying the presence of substantially 20 contiguous nucleotides of the nucleic acid molecule comprising SEQ ID NO. 1 or a complement thereof.

12. A method as claimed in claim 7 step c) utilizes one oligonucleotide primer complementary to 10-12 contiguous nucleotides of SEQ ID NO. 1 or a complement thereof, and one oligonucleotide primer complementary to 10-12 nucleotides of IS900 or a complement thereof.

13. A method as claimed in claim 7, wherein step c) utilizes one oligonucleotide primer complementary to substantially 15 contiguous nucleotides of SEQ ID NO. 1 or a complement thereof; and one oligonucleotide primer complementary to substantially 15 nucleotides of IS900 or a complement thereof.

14. A method as claimed in claim 7, wherein step c) utilizes one oligonucleotide primer complementary to substantially 20 contiguous nucleotides of SEQ ID NO. 1 or a complement thereof, and one oligonucleotide primer complementary to substantially 20 nucleotides of IS900 or a complement thereof.

15. A method as claimed in claim 7, wherein step d) of the method comprises identifying the presence of 10-12 contiguous nucleotides of the nucleic acid molecule comprising SEQ ID NO. 2 or a complement thereof.

16. A method as claimed in claim 7, wherein step d) of the method comprises identifying the presence of at least 15 contiguous nucleotides of the nucleic acid molecule comprising SEQ ID NO. 2 or a complement thereof.

17. A method as claimed in claim 7, wherein step d) of the method comprises identifying the presence of approximately 20 contiguous nucleotides of the nucleic acid molecule comprising SEQ ID NO. 2 or a complement thereof.

18. A method as claimed in claim 7 wherein step c) utilizes one oligonucleotide primer complementary to 12 contiguous nucleotides of SEQ ID NO. 2 or a complement thereof, and one oligonucleotide primer complementary to 10-12 nucleotides of IS900 or a complement thereof.

19. A method as claimed in claim 7, wherein step c) utilizes one oligonucleotide primer complementary to substantially 15 contiguous nucleotides of SEQ ID NO. 2 or a complement thereof; and one oligonucleotide primer complementary to substantially 15 nucleotides of IS900 or a complement thereof.

20. A method as claimed in claim 7 wherein step c) utilizes one oligonucleotide primer complementary to substantially 20 contiguous nucleotides of SEQ ID NO. 2 or a complement thereof, and one oligonucleotide primer complementary to substantially 20 contiguous nucleotides of IS900 or a complement thereof.

21. A method to determine whether a strain of either sheep type or cattle type M. paratuberculosis is present in a sample, comprising:
isolating nucleic acid from said sample; and
identifying the binding of a probe comprising at least 10-12 continuous nucleotides from SEQ ID NO: 2 or a complement thereof.

22. The method of claim 21, wherein said probe comprises at least 15 contiguous nucleotides selected from the nucleic acid comprising SEQ ID NO. 2 or a complement thereof.

23. The method of claim 22, wherein said probe comprises at least 20 contiguous nucleotides selected from the nucleic acid comprising SEQ ID NO. 2 or a complement thereof to determine whether a strain of either sheep type or cattle type M. paratuberculosis is present in a sample.

24. (canceled)

25. A method to distinguish M. paratuberculosis from another strain of the MAI complex which may be present in a sample, comprising identifying the presence of SEQ ID NO:1 and/or SEQ ID NO:2 in a fragment or complement thereof in the sample, wherein the absence of said sequence indicates the absence of M. paratuburculosis and the presence of said sequence indicates the presence of M. paratuburculosis.

26. (canceled)

27. The method of claim 25, wherein the presence of SEQ ID NO:1 is identified.

28. The method of claim 25, wherein the presence of SEQ ID NO:2 is identified.

29. The method of claim 25, wherein the presence of M. paratuberculosis is identified as part of the diagnosis of Johne's disease or Crohn's disease.

30. (canceled)

31. The method of claim 4, wherein the 20 or more contiguous nucleotides are from nucleotides 230-260 of SEQ ID NO:1.


SEQUENCE LISTING (2 sequences)

SEQ ID NO: 1 - DNA, 342 bp, Mycobacterium paratuberculosis
gcttgacaacgtcattgagaatccccttgctacttagctgacctatctacaggctgggtcagcataatcgcagcgtcggacaaggcgtggaggaatcgcgtcgcgacttatgcaactccttcaaaaccggcgtcgcgccgccgtgtgaaacgcaggtcaatccgcaccgggatggtgatcggttcaagtcgtccgaccatgggcggttggggacccggacactcgagacatcacccctagctgatatcagctttagctgatatcatgcggtagtgcggaaatgggaggttgacctgactctcatcgaagcctggatggatgccctcaatgacgttgtcaagc

SEQ ID NO: 2 - DNA, 2306 bp, Mycobacterium paratuberculosis
ttgaagttgccgaaggtgcgaatcgagccgtcggtgtcgaaggcgacggtgtcgatcttggtgtgcggcaggcgcaggaactcgtcgatgaggcccagctcgtccatgatccgcagcgtggtcggatgcacggtgtcgccgcggaagtcgcgcaggaagtcgttgtgcttttccaagacgacgaccgggattccggcccgggcgagcagcagcgcgtgcaccatcccggcgggaccgcccccggcaacgcacacctgagtccggatcacttgcgcaggctacctcccgggtccaaccactaggccgtccaacctcgacggccgctacagagctacacgagccccgcccgagaacatcgagtcatggaactctatcgcttccggcaccgcgcccggcgggatgtcgaacgagacgctcccatccataccgaggccagggttgatgtcttccatccaggtgttgtcgctgagcgaggtcgtcgcgtcaaacttcttgccgtcgatgatcagtttttggttggaagcagagaagctttgggacttgtcgccgatgttcgtcacgcggagcttcacgacgaaaaactcgcccttggcttgctgcaaactgaagaccccttccttagtggccgagcgatcgacaccaagcacttgaaactcgaacttaccgtctcgcaccggggacccagcgggggcgacggcaggccctacatgctttggcttcgtcggcgcgcttgcccttgaggggaccgacgctgccgtgttcgactccgcgacggtcttcttgtcgccgtcgttgccgccgctggcgatggcaatcaggaccaccactacggctccggcgcctaggatccacttcttatcggacttcttctggcggctgggcttcacgggctggccaacgccgggtggcggcgcgggaaatggcgcataactttcggtccacgctgtgccgtcgaagtaccgctggcggctcggattagatggatcgggataccaacccgctgttggctgcgtcatgaagtccccttgctacttagctgacctatctacaggctgggtcagcataaccgcagcgtcggacaaggcgtggaggaatcgcgtcgcgacttatgcaactccttcaaaaccggcgtcgcgccgccgtgtgaaacgcaggtcaatccgcaccgggatggtgatcggttcaagtcgtccgaccatgggcggttggggacccggacactcgagacatcacccctagctgatatcatgcggtagtgcggaaatgggaggttgacctgactctcatcgaagcctggatggatgccctcgatgacgaggagtacgacaacttgatagcagcgctcgagcagttggaggaacacggtccaattacccggcggccgtttgtggacacccttgaagggtcaaggcacccgaacatgaaggagctccgtccgcgccccacgaaagctggagcccacattcgcgtgctattcgccttcgatacccggtcgcgggcgatcatgttgatcgctggagacaaggcgggcaattggtccaagtggtatgccaagcacattcccattgccgacgagctcttcgatgctcaccaaaagcgtttacacaaggcagcagccaaagccaccaatcggaaaccaaggaagggaaagaaacgatgaccaatcttgatgacatgcgccgtcgccgtcccggcaaccggacccgtatcgacgcgatcaaggcggagatggaccgcgaggttgcccagtaccgtctccgcgaactgcgcgaggctgccggctacacgcagacgactctcgctgcggctattggcgtcgggcagaaccgcgtctcgcagatggaacatggcgatctgggcacgagccgggtcgacactctccgcaagtacgtggaagcgactgggggtgagctggaagtgtccgtcaagcgtccggacgggtcacgggttctcctcagcctgtaaagcgcgacgaccgagacccagggcgaaggcttcgagaacggtgtccaggtaccggctgcggcttgtgattctcgggtgccctcggcgctcagaagcagtcgaatgacggggccatcgatacagaatcgacggagaaccgaagcagtacaacagatctcgtcgcacatcggcctcaactcgatcgcggcacagatcaggtaaccgtattcgcggacgtcgccaagaccgactccgatgcaacacgatcacccgtctagcacggctagagtcacctgtcggactgtcgcgcgaccgctgtcgtccaccgaagacgacgaggacaccacggcgaagtaccaccc